(19) United States
(12) Patent Application Publication
(10) Pub. No.: US 2007/0225278 A1
(43) Pub. Date: Sep. 27, 2007

(54) Methods for treating cognitive and other disorders

(75) Inventor: Sharon Rosenzweig-Lipson, East Brunswick, NJ (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 11/726,924

(22) Filed: Mar. 23, 2007

Related U.S. Application Data
(60) Provisional application No. 60/785,654, filed on Mar. 24, 2006.
(60) Provisional application No. 60/851,278, filed on Oct. 12, 2006.

(51) Int. Cl. A61K 31/551; A61K 31/498

(52) U.S. Cl. 514/220; 514/250

(57) ABSTRACT

This invention provides methods and pharmaceutical compositions for treating cognitive disorders such as learning disorders and ADD/ADHD, and other disorders.

### CROSS-REFERENCE TO RELATED APPLICATIONS

This application claims priority to U.S. provisional patent application Ser. No. 60/785,654, filed Mar. 24, 2006, and U.S. provisional patent application Ser. No. 60/851,278, filed Oct. 12, 2006, the entirety of each of which is hereby incorporated herein by reference.

### FIELD OF THE INVENTION

The present invention relates to compounds useful in treating disorders associated with 5HT_{2C} modulation.

### BACKGROUND OF THE INVENTION

Cognition is the ability of one's brain to think, to process and store information, and to solve problems. Cognitive abilities include memory, language, attention, perception, and reasoning. Cognition is a high level of behavior unique to humans. Many cognitive disorders affect the elderly, such as Alzheimer's disease and memory deficit. However, there are also many cognitive disorders that affect children, adolescents, and young adults.

There are a variety of brain abnormalities which prevent infants and children from developing normal social and/or cognitive skills. Disorders of the basic psychological processes can affect the way a child/adolescent learns. Many children/adolescents with learning disabilities have average or above average intelligence. However, learning disabilities may cause difficulties in listening, thinking, talking, reading, writing, spelling, or arithmetic. Such learning disabilities include perceptual handicaps, dyslexia, dyscalculia, dysgraphia and developmental aphasia.

Attention deficit disorders (ADD), also known as attention deficit hyperactivity disorder (ADHD), is a well-known cognitive disorder that affects children and adults alike. It is estimated that between 3% and 8% of all children have ADD. ADD is characterized by symptoms such as hyperactivity, impulsiveness, distractibility and difficulty sustaining attention for periods of time. Symptoms may be different in each person with ADD. Some may have more of a problem with inability to focus, while others may have the most difficult time with impulsiveness. Medications are available to treat ADD, often in the form of stimulants such as Ritalin, Adderal, and Strattera, to name a few. However, there are certain side effects associated with such treatments, including decreased appetite and problems sleeping.

Accordingly, there remains a need to develop treatments for the variety of cognitive disorders affecting patients of all ages.

### SUMMARY OF THE INVENTION

The present invention provides methods for treating a cognitive disorder in a mammal, including methods for treating a learning disorder, an attention deficit disorder, an impulsivity disorder, or a behavioral addiction, among others. In particular, according to the present invention, compounds of formula I: 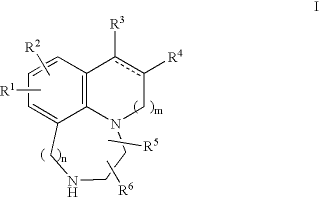
or a pharmaceutically acceptable salt thereof, wherein:
[symbol] designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R,
   which are highly specific agonists, or partial agonists, of the 5HT_{2C} receptor, are useful in the treatment of cognitive and other disorders as described herein.

The present invention provides, among other things, methods of a cognitive disorder by administrating to an individual in need thereof a pharmaceutically effective amount of a compound of formula I. The invention also provides pharmaceutical compositions of compounds of formula I formulated and dosed for treatment of a cognitive disorder, as well as combinations of compounds of formula I with one or more other agents useful in the treatment of cognitive and/or other disorders or diseases suffered by individuals with cognitive disorders. Yet other aspects of the present invention will be clear to those of ordinary skill in the art upon review of the present specification and claims.

### BRIEF DESCRIPTION OF THE DRAWING

FIG. 1 shows the effects of Compound 1 in the sexual function evaluation assay.

FIG. 2 shows the effects of Compound 1 (3-17 mg/kg IP) on reversal of d-amphetamine (4 mg/kg SC) induced deficits in PPI.

FIG. 3 shows the effects of Compound 1 (3-30 mg/kg IP) on reversal of DOI (3 mg/kg IP) induced deficits in PPI.

FIG. 4 shows the effects of Compound 1 (3-30 mg/kg IP) on reversal of MK-801 (0.15 mg/kg SC) induced deficits in PPI.

FIG. 5 shows the effects of Compound 1 on schedule-induced polydipsia.

FIG. 6 shows the effects of Compound 1 on acetylcholine in medial prefrontal cortex.

FIG. 7 shows the effects of Compound 1 on glutamate in medial prefrontal cortex.

FIG. 8 shows the effects of Compound 1 on novel object recognition.

FIG. 9 shows the effect of Compound 1 on impulsive responding in the 5-choice serial reaction time test.

### DETAILED DESCRIPTION OF CERTAIN EMBODIMENTS OF THE INVENTION

1. Compounds

Compounds useful for treating cognitive, and other disorders, according to the present invention include compounds of formula I: 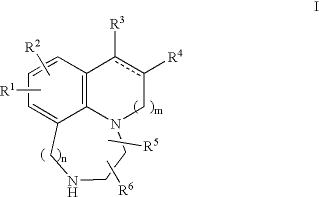
or a pharmaceutically acceptable salt thereof, wherein:
[symbol] designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
   each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R.

As used herein, the term “alkyl” includes, but is not limited to, straight and branched chains such as methyl, ethyl, n-propyl, isopropyl, n-butyl, isobutyl, sec-butyl, or t-butyl.

The terms “halogen” or “halo,” as used herein, refer to chlorine, bromine, fluorine or iodine.

The term “perfluoroalkyl,” as used herein refers to an alkyl group, as defined herein, wherein every hydrogen atom on said alkyl group is replaced by a fluorine atom. Such perfluoroalkyl groups include —CF₃.

The terms “effective amount” and “therapeutically effective amount,” as used herein, refer to the amount of a compound or combination that, when administered to an individual, is effective to treat, prevent, delay, or reduce the severity of a condition from which the patient is suffering. In particular, a therapeutically effective amount in accordance with the present invention is an amount sufficient to treat, prevent, delay onset of, or otherwise ameliorate at least one symptom of a cognitive, or other disorder as described herein.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

The term “patient,” as used herein, refers to a mammal. In certain embodiments, the term “patient” refers to a human.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

The compounds of formula I, as defined above or in classes and subclasses as described herein, have affinity for and agonist or partial agonist activity at the 2C subtype of brain serotonin receptors.

2. Description of Exemplary Compounds:

In certain embodiments, [symbol] designates a single bond. In other embodiments, [symbol] designates a double bond.

In certain embodiments, the R¹ group of formula I is R, OR, halogen, cyano, or —C₁₋₃ perfluoroalkyl. In other embodiments, the R¹ group of formula I is hydrogen, halogen, cyano, —OR wherein R is C₁₋₃ alkyl, or trifluoromethyl. According to another embodiment, the R¹ group of formula I is hydrogen.

In certain embodiments, the R² group of formula I is R, OR, halogen, cyano, or —C₁₋₃ perfluoroalkyl. In other embodiments, the R² group of formula I is hydrogen, halogen, cyano, —OR wherein R is hydrogen, C₁₋₃ alkyl, or trifluoromethyl. According to another embodiment, the R² group of formula I is hydrogen.

According to one aspect of the present invention, at least one of R¹ and R² groups of formula I is —OH. According to another aspect of the present invention, both of the R¹ and R² groups of formula I are —OH.

According to another embodiment, each of the R¹ and R² groups of formula I is hydrogen. According to yet another embodiment, each of the R⁵ and R⁶ groups of formula I is hydrogen.

As defined generally above, the R³ and R⁴ groups of formula I are taken together to form a saturated or unsaturated 4-8 membered ring, wherein said ring is optionally substituted with 1-3 groups independently selected from halogen, —R, or OR. According to one embodiment, the R³ and R⁴ groups of formula I are taken together to form a saturated or unsaturated 5-8 membered ring, wherein said ring is optionally substituted with 1-3 groups independently selected from halogen, —R, or OR. In certain embodiments, the R³ and R⁴ groups of formula I are taken together to form a saturated or unsaturated 5-6 membered ring, wherein said ring is optionally substituted with 1-3 groups independently selected from halogen, —R, or OR.

As defined generally above, n is 1 or 2. Accordingly, the present invention provides a compound of formulae I-a and I-b: 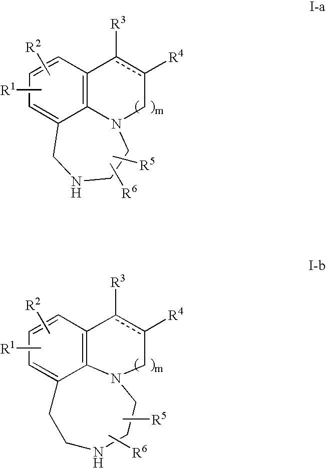
or a pharmaceutically acceptable salt thereof, wherein each of m, R¹, R², R³, R⁴, R⁵, and R⁶ is as defined above for compounds of formula I and described in classes and subclasses above and herein.

As defined generally above, m is 0 or 1. Accordingly, the present invention provides a compound of formulae I-c and I-d: 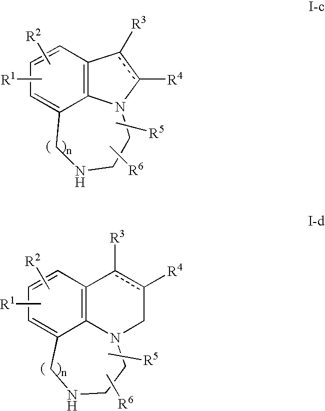
or a pharmaceutically acceptable salt thereof, wherein each of n, R¹, R², R³, R⁴, R⁵, and R⁶ is as defined above for compounds of formula I and described in classes and subclasses above and herein.

In other embodiments, n is 1, m is 1, and the R³ and R⁴ groups of formula I are taken together to form a saturated 5-membered ring and said compound is of formula II: 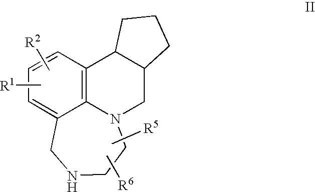
or a pharmaceutically acceptable salt thereof, wherein each of R¹, R², R⁵, and R⁶ is as defined above for compounds of formula I and described in classes and subclasses above and herein.

According to another aspect of the present invention, a compound is provided, wherein n is 1, m is 0, and the R³ and R⁴ groups of formula I are taken together to form a saturated 5-membered ring and said compound is of formula III: 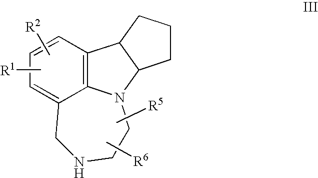
or a pharmaceutically acceptable salt thereof, wherein each of R¹, R², R⁵, and R⁶ is as defined above for compounds of formula I and described in classes and subclasses above and herein.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers.

According to another aspect, the present invention provides a compound of either of formulae I-e or I-f: 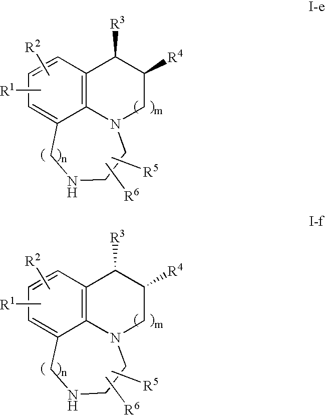
or a pharmaceutically acceptable salt thereof, wherein each of n, m, R¹, R², R³, R⁴, R⁵, and R⁶ is as defined above for compounds of formula I and described in classes and subclasses above and herein.

In certain embodiments, the present invention provides a compound of either of formulae IV or V: 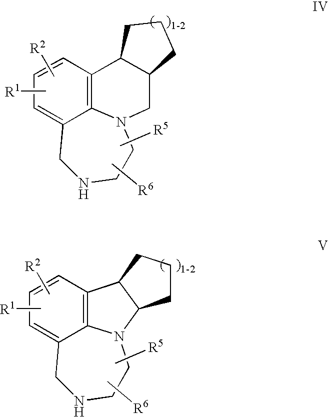
or a pharmaceutically acceptable salt thereof, wherein each R¹, R², R⁵, and R⁶ are as defined above for compounds of formula I and in classes and subclasses as described above and herein.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Exemplary compounds useful for methods of the present invention are set forth in Table 1, below.

TABLE 1
Exemplary Compounds of Formula I

2-bromo-4,5,6,7,9,9a,10,11,12,12a-decahydrocyclopenta[c][1,4]diazepino [6,7,1-ij]quinoline;
2-bromo-4,5,6,7,9,9a,10,11,12,13,14,14a-dodecahydrocyclohepta[c][1,4]diazepino[6,7,1-
ij]quinoline;
2-chloro-4,5,6,7,9,9a,10,11,12,12a-decahydrocyclopenta[c][1,4]diazepino [6,7,1-ij]quinoline;
2-chloro-4,5,6,7,9,9a,10,11,12,13,14,14a-dodecahydrocyclohepta[c][1,4] diazepino[6,7,1-
ij]quinoline;
2-phenyl-4,5,6,7,9,9a,10,11,12,12a-decahydrocyclopenta[c][1,4]diazepino [6,7,1-ij]quinoline;
2-methoxy-4,5,6,7,9,9a,10,11,12,12a-decahydrocyclopenta[c][1,4]diazepino [6,7,1-ij]quinoline;
1-fluoro-4,5,6,7,9,9a,10,11,12,12a-decahydrocyclopenta[c][1,4]diazepino [6,7,1-ij]quinoline;
1-fluoro-4,5,6,7,9,9a,10,11,12,13,14,14a-dodecahydrocyclohepta[c][1,4] diazepino[6,7,1-
ij]quinoline;
1-(trifluoromethyl)-4,5,6,7,9,9a,10,11,12,12a-decahydrocyclopenta[c][1,4] diazepino[6,7,1-
ij]quinoline;
1-fluoro-2-methoxy-4,5,6,7,9,9a,10,11,12,12a-decahydrocyclopenta[c][1,4] diazepino[6,7,1-
ij]quinoline;
1-fluoro-2-methoxy-4,5,6,7,9,9a,10,11,12,13,14,14a-dodecahydrocyclohepta[c][1,4]diazepino[6,7,1-
ij]quinoline;
4,5,6,7,9,9a10,11,12,12a-decahydrocyclopenta[c][1,4]diazepino[6,7,1-ij] quinoline;
4,5,6,7,9,9a,10,11,12,13,14,14a-dodecahydrocyclohepta[c][1,4]diazepino [6,7,1-ij]quinoline;
(−)-4,5,6,7,9,9a10,11,12,12a-decahydrocyclopenta[c][1,4]diazepino[6,7,1-ij] quinoline;
(9aR,14aS)-4,5,6,7,9,9a,10,11,12,13,14,14a-dodecahydrocyclohepta[c][1,4] diazepino[6,7,1-
ij]quinoline; or
(9aS,14aR)-4,5,6,7,9,9a,10,11,12,13,14,14a-dodecahydrocyclohepta[c][1,4] diazepino[6,7,1-
ij]quinoline;
4,5,6,7,9a,10,11,12,13,13a-decahydro-9H-[1,4]diazepino[6,7,1-de]phenanthridine;
1,2,3,4,9,10-hexahydro-8H-cyclopenta[b][1,4]diazepino[6,7,-hi]indole;
1,2,3,4,8,9,10,10a-octahydro-7bH-cyclopenta[b][1,4]diazepino[6,7,1-hi]indole;
(7bS,10aS)-1,2,3,4,8,9,10,10a-octahydro-7bH-cyclopenta[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
6-methyl-1,2,3,4,9,10-hexahydro-8H-cyclopenta[b][1,4]diazepino[6,7,1-hi]indole;
2S)-(rel-7bR,10aR)-2-methyl-1,2,3,4,8,9,10,10a-octahydro-7bH-cyclopenta[b][1,4]diazepino[6,7,1-
hi]indole;
(2S)-(rel-7bR,10aR)-2-methyl-1,2,3,4,8,9,10,10a-octahydro-7bH-cyclopenta[b][1,4]diazepino[6,7,1-
hi]indole;
(2S)-(rel-7bS,10aS)-2-methyl-1,2,3,4,8,9,10,10a-octahydro-7bH-cyclopenta[b][1,4]diazepino[6,7,1-
hi]indole;
(2R)-(rel-7bR,10aR)-2-methyl-1,2,3,4,8,9,10,10a-octahydro-7bH-cyclopenta[b][1,4]diazepino[6,7,1-
hi]indole;
(2R)-(rel-7bR,10aR)-2-methyl-1,2,3,4,8,9,10,10a-octahydro-7bH-cyclopenta[b][1,4]diazepino[6,7,1-
hi]indole;
(2R)-(rel-7bS,10aS)-2-methyl-1,2,3,4,8,9,10,10a-octahydro-7bH-cyclopenta[b][1,4]diazepino[6,7,1-
hi]indole;
rel-(4S,7bS,10aS)-4-methyl-1,2,3,4,8,9,10,10a-octahydro-7bH-
cyclopenta[b][1,4]diazepino[6,7,1-hi]indole;
rel-(4S,7bS,10aS)-4-methyl-1,2,3,4,8,9,10,10a-octahydro-7bH-cyclopenta[b]-
[1,4]diazepino[6,7,1-hi]indole;
rel-(4R,7bS,10aS)-4-methyl-1,2,3,4,8,9,10,10a-octahydro-7bH-
cyclopenta[b][1,4]diazepino[6,7,1-hi]indole;
9-methyl-1,2,3,4,8,9,10,10a-octahydro-7bH-cyclopenta[b][1,4]diazepino[6,7,1-hi]indole;
(7bR,9R,10aR)-9-methyl-1,2,3,4,8,9,10,10a-octahydro-7bH-cyclopenta[b][1,4]diazepino[6,7,1-
hi]indole;
9,9-dimethyl-1,2,3,4,8,9,10,10a-octahydro-7bH-cyclopenta[1,4]diazepino[6,7,1-hi]indole;
(7bR,10aR)-9,9-dimethyl-1,2,3,4,8,9,10,10a-octahydro-7bH-cyclopenta[b][1,4] diazepino[6,7,1-
hi]indole; and
(7bS,10aS)-9,9-dimethyl-1,2,3,4,8,9,10,10a-octahydro-7bH-cyclopenta[b][1,4] diazepino[6,7,1-
hi]indole;
or a pharmaceutically acceptable salt thereof. Another aspect of the present invention provides
the hydrochloride salt of each of the above compounds.

Also, it will be appreciated by those of ordinary skill in the art that reference to a compound herein is intended to include reference to any and all related forms such as polymorphs, hydrates, etc. Also, compounds may be provided as pro-drugs or other forms converted into the active agent during manufacture, processing, formulation, delivery, or in the body.

It will additionally be appreciated that the principles of the present invention apply all radiolabelled forms of the compounds recited herein, including, for example, those where the radiolabels are selected from as ³H, ¹¹C, ¹⁴C, ¹⁸F, ¹²³I, and ¹²⁵I. Such radiolabelled compounds are useful as research and diagnostic tools in metabolism pharmacokinetics studies and in binding assays in both animals and humans.

Compounds of formula I for use in accordance with the present invention may be obtained or produced according to any available means including methods described in detail in WO 03/091250, published Nov. 6, 2003, and in WO 06/052768, published May 18, 2006, the entirety of each of which is hereby incorporated herein by reference.

Without wishing to be bound by any particular theory, the present inventors note that compounds of formula I are highly specific agonists, or partial agonists, of the 5HT_{2C} receptor. The present invention encompasses the recognition that this unique affinity and selectivity displayed by compounds of formula I renders them particularly useful for treating cognitive, and other disorders. The present invention also contemplates that compounds of formula I are associated with a rapid onset of action. In addition, compounds of formula I lack the side-effect of sexual dysfunction.

2. Pharmaceutical Compositions

Compounds of formula I may be administered neat in order to treat a cognitive, or other disorder, in accordance with the present invention. More commonly, however, they are administered in the context of a pharmaceutical composition, that contains a therapeutically effective amount of one or more compound of formula I together with one or more other ingredients known to those skilled in the art for formulating pharmaceutical compositions.

As used herein, the terms “pharmaceutically effective amount” or “therapeutically effective amount” mean the total amount of each active component of the pharmaceutical composition or method that is sufficient to show a meaningful patient benefit, i.e., treatment, prevention or amelioration of a cognitive, or other disorder. When applied to an individual active ingredient, administered alone, the term refers to that ingredient alone. When applied to a combination, the term refers to combined amounts of the active ingredients that result in the therapeutic effect, whether administered in combination, serially or simultaneously.

In certain embodiments of the invention, compounds of formula I are administered with a daily dose in the range of about 0.5 to about 500 mg, or about 1 mg to about 500 mg. Doses may be administered as a single regimen, such as only prior to bedtime or before travel, or as a continuous regimen divided by two or more doses over the course of a day. The dosage levels and other dosage levels herein are for the average human subject having a weight range of about 65 to 70 kg. The skilled person will readily be able to determine the dosage levels required for a subject whose weight falls outside this range, such as children and the elderly.

The dosage of the combination of the invention in such formulations will depend on its potency, but can be expected to be in the range of from 1 to 500 mg of 5-HT_{2C} receptor agonist for administration up to three times a day. In some embodiments, the dose may be in the range of about 10 to 100 mg (e.g. 10, 25, 50 and 100 mg) of 5-HT_{2C} receptor agonist which can be administered once, twice or three times a day (preferably once). However the precise dose will be as determined by the prescribing physician and will depend on the age and weight of the subject and severity of the symptoms.

Additional ingredients useful in preparing pharmaceutical compositions in accordance with the present invention include, for example, carriers (e.g., in solid or liquid form), flavoring agents, lubricants, solubilizers, suspending agents, fillers, glidants, compression aids, binders, tablet-disintegrating agents, encapsulating materials, emulsifiers, buffers, preservatives, sweeteners, thickening agents, coloring agents, viscosity regulators, stabilizers or osmo-regulators, or combinations thereof.

Solid pharmaceutical compositions preferably contain one or more solid carriers, and optionally one or more other additives such as flavoring agents, lubricants, solubilizers, suspending agents, fillers, glidants, compression aids, binders or tablet-disintegrating agents or an encapsulating material. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes or ion exchange resins, or combinations thereof. In powder pharmaceutical compositions, the carrier is preferably a finely divided solid which is in admixture with the finely divided active ingredient. In tablets, the active ingredient is generally mixed with a carrier having the necessary compression properties in suitable proportions, and optionally, other additives, and compacted into the desired shape and size. Solid pharmaceutical compositions, such as powders and tablets, preferably contain up to 99% of the active ingredient.

In certain embodiments, a compound of formula I is provided in a disintegrating tablet formulation suitable for pediatric administration.

Liquid pharmaceutical compositions preferably contain one or more compounds of formula I and one or more liquid carriers to form solutions, suspensions, emulsions, syrups, elixirs, or pressurized compositions. Pharmaceutically acceptable liquid carriers include, for example water, organic solvents, pharmaceutically acceptable oils or fat, or combinations thereof. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators, or combinations thereof. If the liquid formulation is intended for pediatric use, it is generally desirable to avoid inclusion of alcohol.

Examples of liquid carriers suitable for oral or parenteral administration include water (preferably containing additives such as cellulose derivatives such as sodium carboxymethyl cellulose), alcohols or their derivatives (including monohydric alcohols or polyhydric alcohols such as glycols) or oils (e.g., fractionated coconut oil and arachis oil). For parenteral administration the carrier can also be an oily ester such as ethyl oleate and isopropyl myristate. The liquid carrier for pressurized compositions can be halogenated hydrocarbons or other pharmaceutically acceptable propellant.

In certain embodiments, a liquid pharmaceutical composition is provided wherein said composition is suitable for pediatric administration. In other embodiments, the liquid composition is a syrup or suspension.

Liquid pharmaceutical compositions which are sterile solutions or suspensions can be administered parenterally, for example by, intramuscular, intraperitoneal, epidural, intrathecal, intravenous or subcutaneous injection. Pharmaceutical compositions for oral or transmucosal administration may be either in liquid or solid composition form.

In some embodiments of the invention, pharmaceutical compositions are provided in unit dosage form, such as tablets or capsules. In such form, the composition is sub-divided in unit dose containing appropriate quantities of the active ingredient(s). The unit dosage forms can be packaged compositions, for example packeted powders, vials, ampoules, pre-filled syringes or sachets containing liquids. The unit dosage form can be, for example, a capsule or tablet itself, or it can be an appropriate number of any such compositions in package form.

Thus, the present invention also provides a pharmaceutical composition in unit dosage form for a cognitive, or other disorder, in a patient, where the composition contains a therapeutically effective unit dosage of at least one compound of formula I. As one skilled in the art will recognize, the preferred therapeutically effective unit dosage will depend on for example the method of administration. For example, a unit dosage for oral administration often ranges from about 0.5 mg to about 500 mg and more typically from about 1 mg to about 500 mg of the compound of formula I.

The present invention also provides a therapeutic package for dispensing the compounds of formula I to a patient being treated for a cognitive, or other disorder as described herein. In some embodiments, the therapeutic package contains one or more unit dosages of the compound of formula I, a container containing the one or more unit dosages, and labeling directing the use of the package for treating a cognitive, or other disorder, in a patient. In certain embodiments, the unit dose is in tablet or capsule form. In some cases, each unit dosage is a therapeutically effective amount.

3. Other Pharmaceutical Agents

According to the present invention, compounds of formula I may be administered alone to treat one or more cognitive, or other disorders, or alternatively may be administered in combination with (whether simultaneously or sequentially) one or more other pharmaceutical agents useful to treat one or more cognitive, or other disorders, as described herein. Alternatively or additionally, the compounds of formula I may be administered in combination with one or more other pharmaceutical agents useful in the treatment or prevention of one or more other symptoms, disorders, or diseases suffered by the individual in need of treatment of one or more cognitive, or other disorders, as described herein.

An exemplary listing of pharmaceutically active agents that may be administered in conjunction with one or more compounds of formula I in accordance with the present invention can be found in the Physicians' Desk Reference, 55 Edition, 2001, published by Medical Economics Co., Inc., Montvale, N.J. For many of these listed agents, pharmaceutically effective dosages and regimens are known in the art; many are presented in the Physicians' Desk Reference itself.

4. Uses

Methods of this invention are useful for treating one or more cognitive, or other disorders, as described herein, in a patient.

In certain embodiments, the present invention provides a method of treating one or more intellectual deficit disorders comprising administering a compound of the present invention. In other embodiments, such intellectual deficit disorders include dementia, such as dementia of aging, vascular dementia, mild cognitive impairment, age-related cognitive decline, and mild neurocognitive disorder; Alzheimer's disease, and memory deficit, attention deficit disorders (ADD, also known as Attention Deficit Hyperactivity Disorder or ADHD) in both children and adults. In certain embodiments, the present invention provides a method of treating ADD and/or ADHD in a pediatric patient comprising administering to said patient a compound of formula I or pharmaceutical composition thereof.

In other embodiments, the present invention provides a method of treating one or more cognition disorders. According to another aspect, the cognition disorder is a learning disorder. Such learning disorders are known in the art and include autism, dyslexia, Asperger's syndrome, a neurobiological disorder similar to autism and characterized by serious deficits in social and communication skills; specific learning disability, a disorder in one or more of the basic psychological processes involved in understanding or in using spoken or written language, which may manifest itself in an imperfect ability to listen, think, speak, read, write, spell or to do mathematical calculations; dysgraphia, a disorder that causes difficulty with forming letters or writing within a defined space; dyscalculia, a disorder that causes people to have problems doing arithmetic and grasping mathematical concepts; dyspraxia, a problem with the body's system of motion that interferes with a person's ability to make a controlled or coordinated physical response in a given situation; visual perceptual deficit, difficulty receiving and/or processing accurate information from the sense of sight, although there is nothing wrong with vision; and auditory perceptual deficit, difficulty receiving accurate information through auditory means, even though there is no problem with hearing.

In certain embodiments, the present invention provides a method for treating one or more impulsivity disorders (e.g. borderline personality disorder), disruptive behavior disorders, or impulse control disorders. In certain embodiments, the present invention provides a method for treating Tourette's Syndrome (TS), an inherited, neurological disorder characterized by repeated and involuntary body movements (tics) and/or uncontrollable vocal sounds. In other embodiments, the present invention provides a method for treating trichotillomania.

According to another aspect, the present invention provides a method for treating one or more behavioral addictions and addictive disorders. Behavioral addictions and addictive disorders result from the intoxication one senses from the release of brain chemicals (e.g., serotonin, adrenaline, epinepherine, etc.) during certain activities. Such disorders are known in the art and include gambling, sex addiction, eating disorders, spending addiction, rage/anger, workaholism, exercise addiction, risk taking addictions (e.g. kleptomania and pyromania), and perfectionism, to name a few.

In certain embodiments, a compound of the present invention is administered in combination with one or more cognitive improvement agents. Such agents are well known in the art and include donepezil hydrochloride (Aircept™) and other acetylcholinesterase inhibitors; galantamine, neuroprotective agents (e.g., memantine); ADD/ADHD agents (e.g., Ritalin™, Strattera™, Concerta™ and Adderall™), and methylphenidate.

As 5-HT_{2C} modulators, compounds of the present invention are useful for treating a variety of disorders. Such disorders include premenstrual syndrome (PMS), premenstrual dysphoric disorder (PMDD), or late luteal phase syndrome, motion or motor disorders such as Parkinson's disease; chronic fatigue syndrome, anorexia nervosa, disorders of sleep (e.g., sleep apnea), and mutism.

Premenstrual dysphoric disorder, or PMDD, is a severe form of PMS. Like PMS, PMDD typically occurs the week before the onset of menstruation and disappears a few days after. PMDD is characterized by severe monthly mood swings and physical symptoms that interfere with everyday life, especially a woman's relationships with her family and friends. PMDD symptoms go far beyond what are considered manageable or normal premenstrual symptoms.

PMDD is a combination of symptoms that may include irritability, depressed mood, anxiety, sleep disturbance, difficulty concentrating, angry outbursts, breast tenderness and bloating. The diagnostic criteria emphasize symptoms of depressed mood, anxiety, mood swings or irritability. The condition affects up to one in 20 American women who have regular menstrual periods. According to another embodiment, the present invention provides a method for treating one or more symptoms associated with PMDD.

Selective serotonin reuptake inhibitors (SSRIs) are the current preferred method for treating symptoms associated with PMDD. According to another aspect, the present invention provides a method for treating PMDD, or one or more symptoms associated with PMDD, by administering a compound of formula I in combination with an SSRI. In certain embodiments, the SSRI is fluoxetine, venlafaxine, paroxetine, duloxetine, or sertraline.

Inventive methods involve delivery of compounds of formula I via any appropriate route of administration including, for example, oral, buccal, sublingual, rectal, nasal, parenteral, intravenous, or other modes. In general, the compounds may be formulated for immediate, delayed, modified, sustained, pulsed, or controlled-release delivery.

For inventive methods utilizing oral delivery, such delivery may be accomplished using solid or liquid formulations, for example in the form of tablets, capsules, multi-particulates, gels, films, ovules, elixirs, solutions or suspensions. In certain embodiments, the compounds are administered as oral tablets or capsules or neat compound or powdered or granular pharmaceutical formulations. Such preparations may be mixed chewable or liquid formulations or food materials or liquids if desirable, for example to facilitate administration to children, to individuals whose ability to swallow tablets is compromised, or to animals. Examples of oral formulations contained in hard gelatin capsules can include those in which the active compound comprises from about 45% to 50%, by weight, of the formulation. Microcrystalline cellulose comprises from about 43% to about 47%, povidone comprises from about 3% to about 4%, and silicon dioxide and magnesium stearate each comprise from about 0.3% to about 0.7%, each by weight.

Modified release and pulsatile release oral dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, camauba wax, paraffin wax, cellulose acetate phthalate, hydroxypropylmethyl cellulose phthalate, methacrylic acid copolymer and mixtures thereof. Modified release and pulsatile release oral dosage forms may contain one or a combination of release rate modifying excipients. Release rate modifying excipients may be present both within the dosage form i.e., within the matrix, and/or on the dosage form, i.e., upon the surface or coating.

Fast dispersing or dissolving dosage oral formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

For inventive methods utilizing intravenous delivery, such administration may be, for example, intracavernous, intravenous, intra-arterial, intraperitoneal, intrathecal, intraventricular, intraurethral, intrasternal, intracranial, intramuscular or subcutaneous, or via by infusion or needleless injection techniques. For such parenteral administration, the compounds of formula I may be prepared and maintained in conventional lyophylized formulations and reconstituted prior to administration with an intravenously acceptable saline solution, such as a 0.9% saline solution. The pH of the intravenous formulation can be adjusted, as is known in the art, with an intravenous and pharmaceutically acceptable acid, such as methanesulfonic acid.

The compounds of formula I can also be administered intranasally or by inhalation and are conveniently delivered in the form of a dry powder inhaler or an aerosol spray presentation from a pressurized container, pump, spray, atomizer or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurized aerosol, the dosage unit may be determined by providing a valve to deliver a metered amount. The pressurized container, pump, spray, atomizer or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compounds of the invention and a suitable powder base such as lactose or starch.

Aerosol or dry powder formulations are preferably arranged so that each metered dose or “puff” contains from 1 .mu.g to 50 mg of a compound of the invention for delivery to the patient. The overall daily dose with an aerosol will be in the range of from 1 .mu.g to 50 mg which may be administered in a single dose or, more usually, in divided doses throughout the day.

Alternatively, the compounds of formula I can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch, depot or subcutaneous injection. They may also be administered by the pulmonary or rectal routes.

For application topically to the skin, the compounds of formula I can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of formula I may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

### EXAMPLES

Assessment of Effect on Sexual Function

Compound 1, 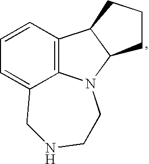
was used to exemplify the effect of compounds of the present invention on sexual function. In summary, male rats were allowed access to sexually receptive females during a single overnight mating session and then administered antidepressants with varying degrees of clinically reported sexual deficits. Following antidepressant treatment, the rats were observed for penile erections in the presence of sexually receptive females that were not accessible for contact, but served as visual, auditory, and olfactory stimuli in the testing area. The details of this assay, and results obtained therefrom, are set forth below.
Animals

Intact male Sprague-Dawley rats (Charles River Laboratories) arrived at 7 weeks of age and were allowed to habituate for one week prior to sexual experience or behavioral testing. Rats were group housed in same-sex colony rooms with food and water provided ad libitum. All animals were maintained under a 12:12 light-dark cycle (lights on at 6:00 am) with behavioral experiments performed during the light hours beginning at noon.

Ovariectomized female Long-Evans rats (Charles River Laboratories) were individually housed and brought into behavioral estrus weekly by subcutaneous injection of 25 ug 17-B estradiol benzoate in 0.1 ml corn oil 48 hours prior to testing, followed by subcutaneous administration of 2.5 mg progesterone in 0.1 ml corn oil 4 to 6 hours before testing.

Establishment of Baseline Conditions

The first set of experiments were conducted to determine conditions under which the baseline level of non-contact penile erections could be increased to allow for a greater window to evaluate potential deficits. Therefore, non-contact penile erections were evaluated in sexually naive and sexually experienced rats in the absence and presence of female stimuli (n=5 pretreatment group).

For subjects assigned into the sexually experienced group, the following procedures were performed. Individual male rats were placed into the home cage of a a sexually receptive female for overnight mating sessions. The cage was a Plexiglas cage measuring 10×12 inches, with food and water ad libitum. Mating pairs were randomly assigned. The interaction between the male and female rat was observed for the first hour. Only males that were observed for intromission within the first hour of paired mating sessions were included in behavioral studies. Following overnight mating sessions, males and females were separated from each other and placed back into same-sex colony rooms where they were group-housed until the time of behavioral testing.

To determine the effects of female stimuli on non-contact penile erections, sexually receptive females were brought into the testing room 30 minutes prior to the start of behavioral testing in order to provide female visual, olfactory, and auditory stimuli for males. The testing arena for observing non-contact penile erections in male rats consisted of an empty rat size Plexiglas cage (11×10 inches) containing no bedding, with an aerated plastic lid and a total ceiling height of 12 inches. Males were observed for non-contact penile erections in individual test cages. A penile erection consisted of observation of the male rat in a hunched position grasping the penis with the forepaws and followed by a series of pelvic thrusts. The number of penile erections were quantified over a 30 minute observation session.

Subjects assigned to the nonstimuli group were observed in the same testing arena as described above, however there were no female rats present in the testing room.

Administration of Compounds

Following sexual experience (as described above), male subjects (n=8 per group) were administered either 10 mg/kg/day fluoxetine or saline vehicle for 14 days. On the 14th day, fluoxetine treatment was co-administered with either saline or apomorphine (0.1-0.3 mg/kg, sc) or yohimbine (0.1-0.3 mg/kg, sc) or sildenafil (0.1-0.3 mg/kg, sc) in separate experiments. Non-contact penile erections were evaluated in 30 minute test sessions in the presence of sexually receptive female rats.

Fluoxetine HCl (Tocris), desipramine (Sigma, St. Louis, Mo.) and bupropion HCl (Toronto Research Chemicals, Canada) were dissolved in 0.9% sterile saline vehicle and injected intraperitoneally (i.p.) once daily. On the day of behavioral testing, antidepressants were administered intraperitoneally (i.p.) as one hour pretreatments. For reversal experiments, apomorphine (Sigma, St. Louis, Mo.), yohimbine (Sigma, St. Louis, Mo.) or sildenafil (Toronto Research Chemicals, Canada) were used. Apomorphine was dissolved in 0.9% sterile saline vehicle and injected subcutaneously (s.c.). Yohimbine was dissolved in dH2O facilitated with 1N HCl, followed by addition of 1N NaOH for titration (pH=7) and administered intraperitoneally (i.p.). Sildenafil was dissolved in 0.9% sterile saline vehicle and administered intraperitoneally (i.p.). With the exception of fluoxetine which was administered in a dose volume of 2 ml/kg, all other test compounds were administered in a dose volume of 1 ml/kg. On the day of behavioral testing, both fluoxetine and the reversal agent (apomorphine, yohimbine, or sildenafil) were co-administered as 0 minute pretreatments.

Compound 1 was administered at 3 and 10 mg/kg, sub-cutaneously as a solution in either water or saline.

Statistical Analysis

Data are expressed as mean number of non-contact penile erections per treatment group, calculated as % of vehicle control±S.E.M. For statistical analysis, data were subjected to one-way analysis of variance (ANOVA) with a least significant difference (LSD) post-hoc test as appropriate. Statistical significance was achieved when p<0.05.

Effects of Sexual Experience or Inexperience in the Absence or Presence of Female Stimuli on Non-Contact Penile Erections in Male Rats

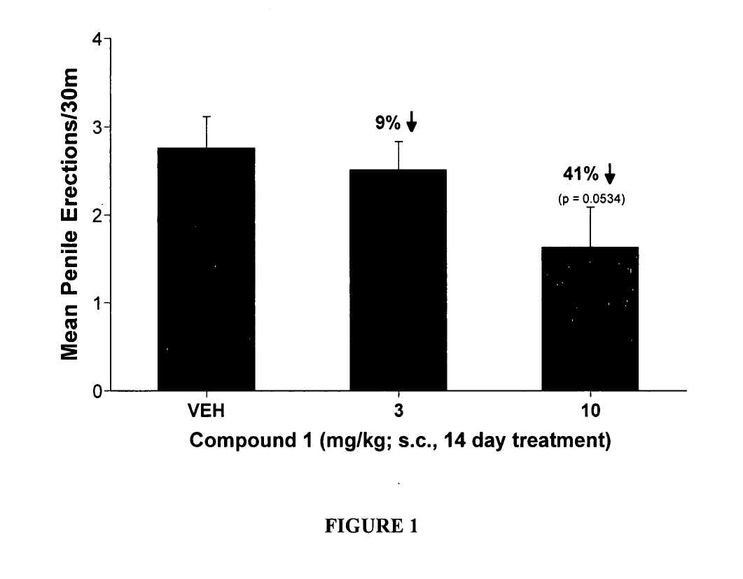

Sexually naive rats evaluated for non-contact penile erections in the absence of female stimuli exhibited a mean of 1.33±0.33 non-contact penile erections as presented in FIG. 1. The presence of female stimuli or an overnight sexual experience did not significantly elevate the number of non-contact penile erections. However, sexually experienced males in the presence of female stimuli resulted in a 58% increase in the number of non-contact penile erections to 3.17±0.75 (p<0.05) relative to sexually naive rats in the absence of female stimuli (F[3,23]=1.77; p=0.186). The increased baseline level of non-contact penile erections allows for a sufficient window to evaluate potential deficits and all additional studies were conducted using sexual experienced males in the presence of female stimuli.

The dopamine and norepinephrine reuptake inhibitor (D/NRI), bupropion, the tricyclic antidepressant (TCA), desipramine, and the selective serotonin reuptake inhibitor (SSRI), fluoxetine reduced the number of penile erections, 9%, 43%, and 72%; respectively, relative to vehicle treated animals. This rank order of the compounds propensity for reducing the number of penile erections is comparable to the rank order of the compounds' ability to produce sexual dysfunction clinically. Additionally, compounds with varying mechanisms of action used clinically to treat antidepressant-induced sexual deficits were effective in reversing the deficits produced by chronic fluoxetine treatment in this model.

Table 2, below, summarizes the dose at which antidepressant-like effects are observed, the dose at which sexual dysfunction occurs, and the therapeutic window.

TABLE 2

| | Bupropion | Desipramine | Fluoxetine | Compound 1 |
|---|---|---|---|---|
| Antidepressant-like | 20 | 10 | 10 | 0.3 |
| effects | | | | |
| (MED mg/kg) | | | | |
| Sexual dysfunction | >20 | 10 | 10 | ≧10 |
| (MED mg/kg) | | | | |
| Therapeutic window | >1 | none | none | ≧30-fold |

These data show, as set forth in Table 2, that the dose of DMI or fluoxetine that produces an antidepressant-like effect is equal to the dose producing sexual side-effects. For Compound 1, the dose producing sexual side-effects is at least 30 times the dose required for an antidepressant-like effect. For bupropion the window is about 2-fold.

### Example 2

Assessment of Effect on Attention

Compound 1, 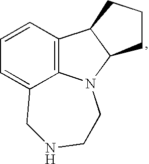
was used to exemplify the effect of compounds of the present invention on attention. The objective of this study was to characterize the ability of Compound 1 to attenuate pharmacological disruptions of PPI produced by a variety of disrupting agents that have been shown to be differentially sensitive to the effects of typical and atypical antipsychotics. Data generated within our lab has shown that the typical antipsychotics haloperidol, a selective D₂ antagonist, will attenuate a d-amphetamine induced deficit in PPI, while the atypical antipsychotics risperidone, olanzapine and clozapine, will attenuate deficits in PPI produced by DOI and MK-801, as well as those caused by d-amphetamine. The results of the current studies with Compound 1 demonstrate an ability to attenuate both d-amphetamine and DOI induced gating deficits, in a dose range that has no effect on baseline PPI when Compound 1 is administrated alone. Although we were not able to demonstrate an ability of Compound 1 to attenuate an MK-801 induced deficit in PPI, Compound 1 did attenuate the increased startle reactivity induced by MK-801, and this effect is similar to that seen with other antipsychotics. The pre-pulse data presented in this Example, support an atypical antipsychotics-like profile for this compound.
Introduction

Pre-pulse inhibition (PPI) refers to the attenuation in startle response that occurs when a weak, non-startling stimulus (the pre-pulse) is paired with a higher intensity, startle-eliciting pulse. PPI is a cross-species phenomenon, one that can be observed in both humans and rodents, and it is widely reported to be deficient in schizophrenia patients. It has been theorized that deficits in the gating of sensory information contribute to both the positive symptomatology, and the cognitive fragmentation, which are hallmarks of this disease. In the rat, PPI can be disrupted by the systemic administration of dopamine agonists, serotonin agonists, as well as glutamate antagonists. The reversal of these deficits by pharmacological means is proposed to be a model for predicting anti-psychotic activity in novel compounds. Typical and atypical antipsychotics both treat psychotic symptoms, but atypical antipsychotics have a reduced extrapyramidal motor symptom liability. Typical antipsychotics, such as haloperidol, which are primarily selective D₂ antagonists, are efficacious in reversing PPI disruptions elicited by dopamine agonists in animals. Atypical antipsychotics, by virtue of their activity at multiple receptors, should block not only dopamine induced deficits in PPI, but those elicited by other classes of disrupting agents. Published data suggests that PPI deficits in schizophrenics may be influenced by treatment medication, with PPI effects of atypical antipsychotics being differentiated from those of typicals, depending on experimental design. Typical (haloperidol) and atypical (risperidone, olanzapine and clozapine) antipsychotics show a differential ability to reverse pharmacologically induced PPI deficits, with all of the atypicals showing efficacy to reverse or attenuate amphetamine (an indirect dopamine agonist), DOI (a 5-HT_{2A} agonist) and MK-801 (a non-competitive NMDA antagonist) induced PPI deficits, while the typical anti-psychotic haloperidol was effective only against amphetamine PPI deficits. The objective of this study was to evaluate the effects of Compound 1, a potent and selective 5-HT_{2C} agonist, in a model where typical and atypical antipsychotics show differential effectiveness in normalizing various pharmacologically induced deficits in the sensorimotor gating response.

Materials

Animals: For all studies, male Sprague Dawley CD rats (Charles River) weighing 250-400 g were used. Rats were group housed and allowed ad-lib access to food and water. A 12-h light/dark cycle was imposed with the lights on period-between 0600 and 1800 hours. All PPI testing occurred during the lights-on phase, typically between 0800 and 1300 hours.

The animals used in this study are listed in Table 3.

TABLE 3
Animal Subject Summary

| Category | Description |
|---|---|
| Species: | Rat |
| Strain: | Sprague Dawley CD |
| Source: | Charles River |
| No. of Animals Per Group: | 8 |
| Total No. of Animals: | 40-48 per experiment |
| Age and Sex: | Unknown and Male |
| Weight: | Approximately 300 g |
| Date Received: | Varies, typically 2 weeks prior to study |
| Acclimation Period: | 1 week |

Drugs: Compound 1 was dissolved in 0.25% Tween 80 and injected in a volume of 2 ml/kg. MK-801 (S-1319-A-2 or S-1319-A-10), DOI and d-amphetamine (S-204-A-2) were all dissolved in saline and injected in a volume of 1 ml/kg. All doses refer to active moiety.

Methods

Test Equipment Each testing chamber (SR-LAB system, San Diego Instruments) consisted of a Plexiglas cylinder (8.8 cm in diameter) mounted on a frame and held in position by four metal pins to a base unit. Movement of the rat within the cylinder was detected by a piezoelectric accelerometer attached below the frame. A loudspeaker mounted 24 cm above the cylinder provided background white noise, acoustic noise bursts and acoustic pre-pulses. The entire apparatus was housed in a ventilated enclosure (39×38×56 cm). Presentation of acoustic pulse and pre-pulse stimuli were controlled by the SR-LAB software and interface system, which also digitized, rectified and recorded the responses from the accelerometer. Mean startle amplitude was determined by averaging 100, 1 ms readings taken from the beginning of the pulse stimulus onset. For calibration purpose, sound levels were measured with a Quest sound level meter, scale “A”, with the microphone placed inside the Plexiglas cylinder.

Test Sessions Test sessions began when the rats were placed in the startle chambers for a 5-min acclimation period with a 64 dB (A) background of white noise. After the acclimation period, rats were exposed to four types of stimuli. The startle-eliciting stimulus was a 20-ms broad band burst at a sound pressure level of 120 dB (A). Three different intensities of auditory pre-pulse stimuli were utilized. These consisted of a 69, 74 or 79 dB (A), 20-ms broadband burst which was presented 100-ms (onset to onset), prior to the startle pulse. These four trial types were presented against a constant 64 dB (A) background of white noise. A test session consisted of an initial pulse stimulus, followed by 15 sequences of the four stimulus types, presented in pseudorandom order, for a total of 61 trials. Inter-trial intervals averaged 15 s.

Pretreatment Intervals Compound 1 was administered 30 min prior to testing via an IP route of administration. MK-801 (0.15 mg/kg) and d-amphetamine (4 mg/kg) were administered 10 min prior to testing via a SC route of administration, while DOI (3 mg/kg) was administered 15 min prior to testing using an IP route of administration.

Evaluation of Results

Startle amplitude was defined as the mean value of pulse alone trials. To evaluate the effect of drug treatment on startle response, data from the pulse alone trials was analyzed using one-factor ANOVA (one-way randomized block design), followed by a least significant difference (LSD) post-hoc test (comparison was made to vehicle/disrupting agent control, and vehicle control in Compound 1 alone study). Pre-pulse inhibition was defined as 100−[(startle amplitude on pre-pulse trials/startle amplitude on pulse alone trials)×100]. Although data for gating at three different pre-pulse intensities was generated, an averaged gating score across all pre-pulse intensities was calculated and this was analyzed by one factor ANOVA with repeated measures (one-way randomized block design). This was followed by a LSD post-hoc test (comparison was made to vehicle/disrupting agent control, and vehicle control in Compound 1 alone study). The criterion for significance for alterations in both startle amplitude and PPI was set at P<0.05.

Results and Discussion

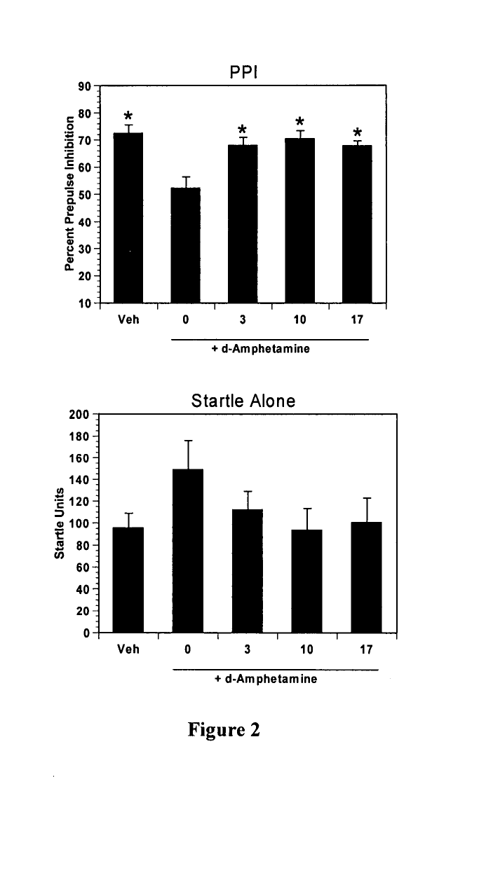

Compound 1 (3-17 mg/kg) significantly attenuated (FIG. 2) an amphetamine induced deficit in PPI [F(4,34)=5.913, P=0.0010] at all doses tested, without having a significant effect on startle alone responding [F(4,34)=1.175, P=0.3394].

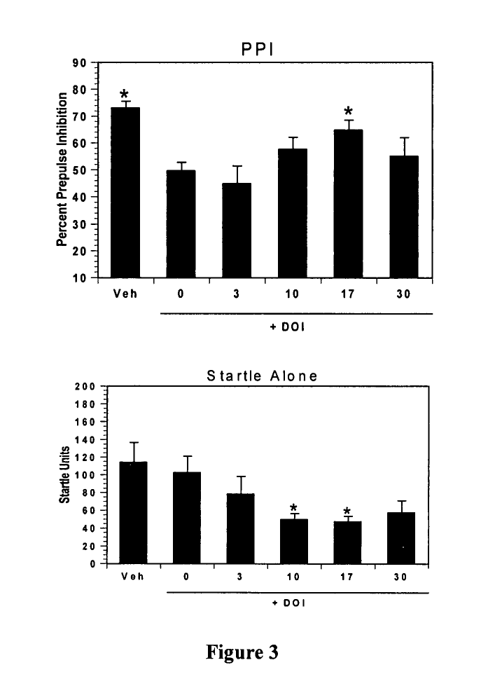

When tested against a DOI induced deficit, Compound 1 (3-30 mg/kg) significantly attenuated the disruption in gating [F(5,60)=5.612, P=0.0002] at a dose of 17 mg/kg (FIG. 3). There was a significant decrease in startle intensity [F(5,69)=3.423, P=0.0080) in those animals receiving the 10 and 17 mg/kg dose of Compound 1 relative to a vehicle/DOI control.

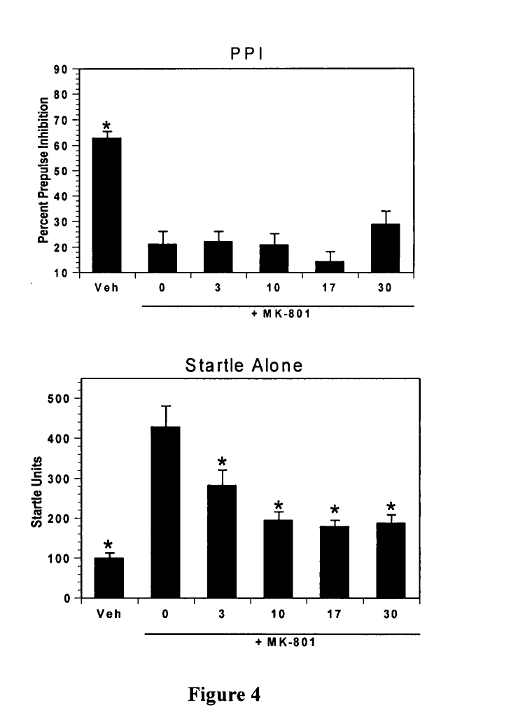

Against the highest dose of MK-801 test (0.15 mg/kg), Compound 1 (3-30 mg/kg) did not show an ability to significantly attenuate the MK-801 induced disruption in gating [F(5,79)=21.078, P<0.001] at any dose tested (FIG. 4), but did significantly reduce the increase in startle produced by MK-801 [F(5,79)=15.456, P<0.001], at all doses tested.

### Example 3

Assessment of Effect on Compulsivity/Impulsivity

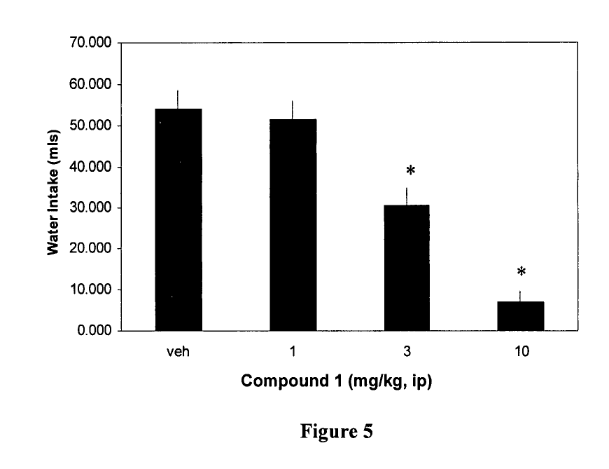

Compound 1, 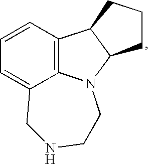
was used to exemplify the effect of compounds of the present invention on compulsivity/impuslivity using the Schedule-Induced Polydipsia assay in rats. The objective of this study was to characterize the effect of Compound 1 (1-10 mg/kg, ip) on schedule-induced polydipsia in rats. Compound 1 produced dose-dependent decreases in adjunctive drinking following ip administration with an MED of 3 mg/kg and an ED₅₀ value of 3.16 mg/kg, ip (95% CI: 2.03-4.92 mg/kg, ip; FIG. 5). These results suggest that Compound 1 may be an effective treatment for obsessive-compulsive disorder and impulsivity disorders.
Introduction

Compound 1 is a potent full agonist at the 5-HT_{2C} receptor (Ki=3 nM; EC₅₀=8 nM). Compound 1 represents a novel therapeutic approach to the treatment of schizophrenia and depression. Additionally, several lines of evidence suggest that 5-HT_{2C} agonists may be effective treatments for obsessive-compulsive disorder. For example, 5-HT_{2C} agonists have been shown to be effective in animal models of compulsive behavior such as schedule-induced polydipsia (SIP), 8-OH-DPAT-induced scratching in squirrel monkeys, marble burying, and excessive eating of palatable foods. Additionally, 5-HT_{2C} knockout mice exhibit compulsive-like behaviors.

The present studies were conducted to evaluate the effects of Compound 1 in a SIP model in rats. SIP is a model of obsessive-compulsive disorder. In this model, a food pellet is delivered once per minute for a two-hour period. A water bottle is available in the chamber. Under this schedule, water intake is tremendously increased relative to animals that receive 120 food pellets at the start of a session and are given two hours to eat and drink. This excessive manifestation of a normal behavior (drinking) provides face validity to the model. Moreover, since the animals are not water deprived, the adjunctive drinking does not serve a physiological function. Most importantly, clinically effective drugs for the treatment of obsessive-compulsive disorder decrease adjunctive drinking.

Materials and Methods

Animals: Male Sprague-Dawley rats weighing 275-325 g were individually housed in wire hanging cages. Each cage was equipped with a water spigot attached to an automated watering system which allow free access to water at all times. Body weights were reduced gradually to 85% of baseline weights by restricting post-session feeding.

Methods Rats were placed in an operant chamber (Med Associates) equipped with a pellet dispenser, a food receptacle, a houselight and a water bottle with 100 ml of water. Houselights were turned on in the chamber and a food pellet (Bioserve, 45 mg) was delivered on a fixed-time 60 sec schedule, such that one pellet was delivered each minute during a 2 hour experimental session. At the end of the session, water intake was measured.

Experiments to control for schedule and baseline water intake were conducted by placing 120 pellets in the food receptacle at the start of a 2-hour experimental session. Houselights were turned on, but no additional food pellets were delivered. At the end of the session, water intake was measured.

Compound Administration Compound 1 was dissolved in 0.9% saline and was administered ip in a volume of 1 ml/kg immediately the 2-hour experimental session. Dose calculations were based on active moiety.

Evaluation of Results Data were analyzed using a one-way ANOVA. Post hoc tests comparing vehicle to doses of Compound 1 were conducted using contrasts in a least squares model. An ED₅₀ value (dose decreasing polydipsic intake to 50% of vehicle values) was calculated using nonlinear regression models.

### RESULTS

Following administration of saline, rats drank 54.0±4.6 ml of water during the 2 hour experimental session during which a food pellet was delivered once per minute. Compound 1 (1-10 mg/kg, ip) produced dose-dependent decreases in excessive water intake in the schedule-induced polydipsia procedure (F(3,39)=28.53; p<0.001). Posthoc tests revealed that 3mg/kg (−43%; p=0.0003) and 10 mg/kg (−87%; p<0.0001) produced significant decreases in excessive water intake. The ED₅₀ value was 3.16 mg/kg, ip (95% CI: 2.03-4.92 mg/kg, ip). At the highest dose, 3 of 10 rats did not eat all of their food pellets during the 2-hour session.

The present Example demonstrates that Compound 1 produces dose-dependent decreases in adjunctive drinking following ip administration with an MED of 3 mg/kg and an ED₅₀ value of 3.16 mg/kg, ip (95% CI: 2.03-4.92 mg/kg, ip). The anti-OCD-like effects of Compound 1 on SIP occurred following an acute administration. In contrast, the effects of serotonin reuptake inhibitors require chronic administration in this model.

### Example 4

Assessment of Effect on Acetylcholine Levels

Compound 1, 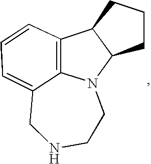
was used to exemplify the effect of compounds of the present invention on rat brain acetylcholine and glutamate using in vivo microdialysis, and to evaluate the pro-cognitive effects of Compound 1 in rat novel object recognition (NOR).

Compound 1 is a selective 5-HT_{2C} receptor agonist which is effective in animal models predictive of antidepressant- and antipsychotic-like activity. Furthermore, Compound 1 was previously reported to increase glutamate in the medial prefrontal cortex, suggesting a potential pro-cognitive effect. To determine whether Compound 1 may cause further neurochemical changes consistent with pro-cognitive effects, Compound 1 was tested for its effects on acetylcholine and glutamate in the medial prefrontal cortex of freely moving rats. Compound 1 was also tested in the rat novel object recognition (NOR) task in order to determine whether it modified cognitive function in a rodent model of learning and memory.

### MATERIALS AND METHODS

Animals: Adult male Sprague-Dawley rats (Charles River, Wilmington, Mass.) weighing 280-350 g at the time of surgery were used for all microdialysis experiments. Adult male Long-Evans rats (Charles River, Wilmington, Mass.) weighing 180-220 g were used for NOR studies. Rats were housed individually for recognition memory studies. All animals had free access to food and water and were group housed in the AAALAC-accredited facility that was maintained on a 12 h light dark cycle (lights on at 0600 h) for at least 1 week prior to surgery or behavioral testing. All in vivo studies were performed in accordance to the ‘Guide for the Care and Use of Laboratory Animals’ as adopted and promulgated by the National Institutes of Health (Pub. 85-23, 1985). The animals used in microdialysis are listed in Table 4.1. The animals used in NOR are listed in Table 4.2.

TABLE 0-1
Test Systems for Microdialysis

| | |
|---|---|
| Species: | Rat |
| Strain: | CD |
| Source: | Charles River |
| No. of Animals Per Group: | 6-8 |
| Total No. of Animals: | 80 |
| Age and Sex: | 60-75 days old; Male |
| Weight: | 250-400 g |
| Acclimation Period: | ≧6 days |

TABLE 0-2
Test Systems for NOR

| | |
|---|---|
| Species: | Rat |
| Strain: | Long-Evans |
| Source: | Charles River |
| No. of Animals Per Group: | 10-15 |
| Total No. of Animals: | 68 |
| Age and Sex: | 50-60 days old; Male |
| Weight: | 180-220 g |
| Acclimation Period: | ≧6 days |

Stereotaxic Surgery Following induction of anesthesia with 3% halothane (Fluothane; Zeneca, Cheshire, UK), animals were secured in a stereotaxic frame with ear and incisor bars (David Kopf, Tujunga, Calif.). Anesthesia was maintained by continuous administration of halothane (1-2%) while a microdialysis guide cannula (CMA/12, CMA Microdialysis, Stockholm, Sweden) was implanted above the medial prefrontal cortex (AP: 3.2 mm ML: −0.6 mm DV: −3.8 mm). The guide cannula was secured to the skull using dental acrylic (Plastics one, Roanoke, Va., USA) and two small stainless-steel screws (Plastics one, Roanoke, Va., USA). Following surgery, animals were individually housed in Plexiglas cages (45 cm sq.), with free access to food and water. The following day rats were used in microdialysis experiments.

Microdialysis Microdialysis probes (CMA 12/02; CMA Microdialysis, Stockholm, Sweden) were equilibrated according to manufacturer's specifications. Microdialysis probes were perfused with artificial cerebrospinal fluid (aCSF: 125 mM NaCl, 3 mM KCl, 0.8 mM MgCl₂, 1.85 mM CaCl₂, 1.54 mM Na₂HPO₄ and 0.225 mM NaH₂PO₄; pH 7.4) prior to insertion in the guide cannula. The microdialysis probe was then inserted via the guide cannula into the dorsal hippocampus or medial prefrontal cortex and perfused with aCSF at a flow rate of 0.5 μl/min. A 3 h stabilization period was allowed following probe insertion before dialysate sampling was initiated. Samples were collected every 40 min for acetylcholine analysis and immediately frozen on dry ice after collection. After baseline samples were collected (2 h), rats were dosed with Compound 1 or vehicle (t=0). Following dosing, dialysis samples were collected for 200 min. At the end of the experiment, animals were euthanized and probe placement was verified histologically. Data from animals with incorrect probe placement were discarded.

Analysis of Acetylcholine Dialysate levels of acetylcholine were determined using LC/MS/MS. The following conditions and equipment were used:

Instrument: Waters Micro mass spectrometer coupled with Agilent HP 1100

Column: Supelco LC-SCX, 2.1×150 mm, 5 mM

Mobile phase: A: 60/20/20 of H2O/buffer/ACN; B: 20/80 buffer/ACN

Buffer: 79.5 mM ammonium acetate, 63.5 mM ammonium formate, pH 4.0

Ionization mode: (+) ESI

MRM: Acetylcholine (ACh): 146 [symbol] 87 Cone=20 CID=15 eV

Iso-ACh: 146 [symbol] 87 Cone=20 CID=15 eV
(3-carboxypropyltrimethylammonium)
IS: b-methyl-ACh: 160 [symbol] 101 Cone=20, CID=15 eV

Acetylcholine data were quantified using peak area against an internal standard and acquired using Masslynx software (Micromass, Beverly, Mass., USA). The mean of the concentration of baseline samples was calculated and denoted as 0%. All sample values were expressed as a percent change from this pre-injection mean baseline value (% change from baseline). Neurochemical data, excluding pre-injection values, were analyzed by a two-way analysis of variance (ANOVA) with repeated measures (time). All statistical analyses were performed using SAS (v 1.03) within Excel® (Microsoft).

High Performance Liquid Chromatography (HPLC) Analysis

Dialysate (10 μL) f was collected and analyzed for extracellular glutamate concentrations. HPLC methods were conducted using the following methods: 10 μL dialysate containing glutamate was separated by HPLC. These units consisted of two Jasco PU-980 pumps (Jasco Ltd, Essex, U.K) as the gradient, a BAS sentinel autosampler (BAS) and a Jasco PF-920 fluorometer with excitation wavelength of 448 nm. The emission wavelength was 485 nm. Mobile phase A was 0.05 M acetate buffer (pH 6.5) with 20% methanol (V:V) and mobile phase B was a 0.05 M acetate buffer (pH 6.5) with 80% methanol (V:V). The gradient consisted of a linear transition from 80% mobile phase A to 0% in 18 minutes. The column was allowed to re-equilibrate for 10 min prior to each injection. Each sample was diluted 1:1 with normal Krebs solution containing 2.5 μM alpha-aminoadipic acid ((AA; final concentration 1.25 μM; internal standard). Samples containing (AA were derivatized with naphthalene 2,3-dicarboxaldehyde (NDA). Samples or standards were mixed with 30 mM boric acid buffer (pH 9.5) containing 20 μM cyanide, and 30 μM NDA in methanol (1:1:0.25; sample: borate: NDA) and were allowed to react for 10 minutes at 10° C. prior to fluorometric detection. Data were acquired using the Atlas software package (Labsystems, Gulph Mills, Pa.) for the PC.

Statistical Analysis of Results

The fmol concentrations of glutamate during the baseline samples were averaged and this value was denoted as 100%. Subsequent sample values were expressed as a percentage of this preinjection baseline value (% of control). Neurochemical data, excluding preinjection values, were analyzed by a two-way analysis of variance (ANOVA) with repeated measures (time). Post-hoc analyses were made using the Bonferroni/Dunns adjustment for multiple comparisons. All statistical calculations were performed using the Statview software application Abacus Concepts Inc., Berkeley, Calif.) for the PC.

Novel Object Recognition

Novel object recognition (NOR) training and testing was performed in a circular field (diameter ˜70 cm, 30 cm high) constructed out of plastic and containing bedding. The field was surrounded by black curtains to mask extra-field cues and was located in a dimly lit room (˜10 lux at the level of the area) in the presence of white noise (˜65 dB). Animal performance was tracked by video and monitored by an experimenter located outside of the testing room. Objects, constructed with Duplo (Lego), were placed on the arena floor in one of four locations spaced evenly around the field approximately 10 cm from the field's edge. To avoid possible olfactory cues multiple copies of the objects were used throughout the study and were cleaned with a 30% ethanol solution between animals. Rats displayed no preference or aversion to either of the objects and spent equivalent amount of time exploring objects if both were presented simultaneously (data not shown).

The visual recognition task was divided into 3 sessions—habituation, a sample trial and a choice trial. During habituation the animals were placed into the field containing 2 identical yellow cubes (˜10×10×10 cm) and were allowed to explore the field for ten minutes. Following habituation rats were returned to their home cage. One day after habituation, animals were dosed with drug and following the pretreatment interval the sample trial was initiated. During the sample trial, rats were allowed to explore the field, now containing two identical objects located at opposing compass points, for 5 minutes. The amount of time exploring the objects was recorded for the entire trial. Exploration was defined as orientation toward the object with the nose of the rat within 2 cm of the object. Following the sample trial rats were returned to their home cages for the 48 hour retention interval and then tested in the choice trial for recognition memory. The choice trial consisted of a 5 minute exploration of the field containing both a familiar, previously explored, object and a novel object with an investigator again recording contact time. The location of the objects, counterbalanced across treatment groups, remained constant for each animal during the habituation, sample and choice trials.

The effect of treatment on object exploration during the sample trial was examined using a one-way ANOVA on total contact time followed by Fisher's LSD group mean pairwise comparisons. The amount of time exploring the novel and familiar objects across treatment groups was analyzed using a repeated measures ANOVA followed by Fisher's LSD post hoc comparisons. Significantly more time spent exploring the novel object than the familiar one during the choice trial represents intact recognition memory for that treatment group. Vehicle control and untreated animals show no significant differences between familiar and novel object exploration following the 48 hour delay indicating no memory for the sample trial.

Compound 1 (0.3-10 mg/kg) was administered orally 60 minutes prior to the sample trial to determine whether it could improve retention in the task.

### RESULTS AND DISCUSSION

Effect of Compound 1 on Acetylcholine in Medial Prefrontal Cortex

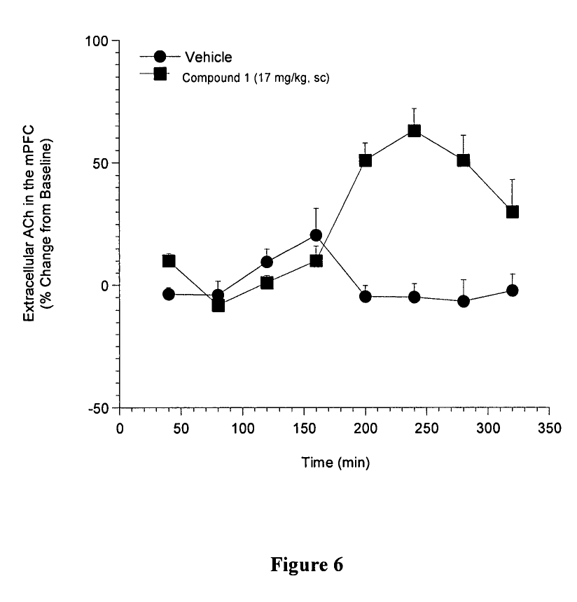

Administration of Compound 1 (17 mg/kg s.c.) produced a significant increase in acetylcholine in medial prefrontal cortex (F1,14=12.03; P=0.004). The maximal increase was 63% with respect to baseline (FIG. 6).

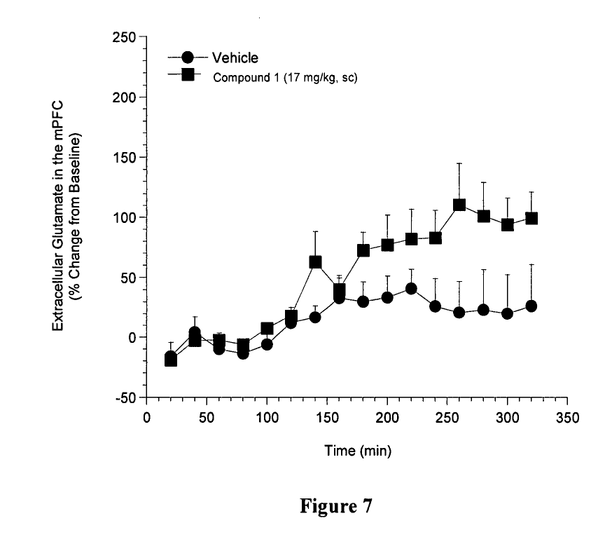

FIG. 7 shows the effect of Compound 1 (17 mg/kg, s.c.) on extracellular glutamate levels in the rat mPFC. We found that acute administration of the selective 5-HT_{2C} agonist significantly (F (1,15)=4.76, P=0.0454) elevated glutamate levels (210%) compared to vehicle-treated animals (FIG. 7).

Novel Object Recognition

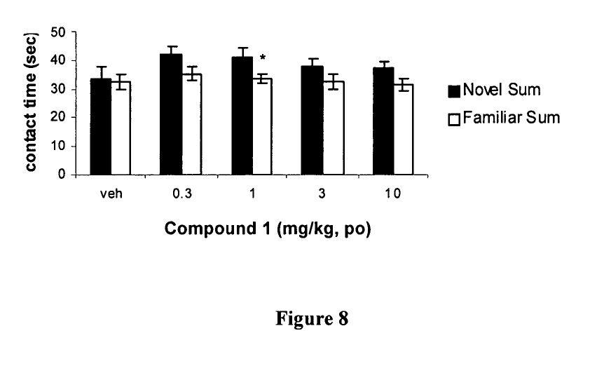

Treatment with 1 mg/kg of Compound 1 resulted in significantly greater time spent exploring the novel than the familiar object demonstrating enhanced retention of the previous learning experience (FIG. 8). Vehicle-treated animals displayed no preferential exploration of the two objects suggesting a natural decay in their original memory for the original object. The maintenance of a significant difference between novel and familiar object exploration in the 1 mg/kg dose group, similar to what is observed in control animals after a short (i.e. 1 hour) delay suggests an enhanced retention of the recognition memory. No significant effect of Compound 1 on exploration during the sample trial was observed at this dose (data not shown).

### CONCLUSION

The 5-HT_{2C} agonist, Compound 1 produces increases in acetylcholine and glutamate in the medial prefrontal cortex a brain area implicated in cognitive function. These data indicates a pro-cognitive neurochemical profile for Compound 1. Furthermore, Compound 1 was demonstrated to be active in the rat novel object recognition model using a time-induced deficit in memory performance.

### Example 5

Effectiveness of Compound I on Impulsive Responding

5-Choice Serial Reaction Time Test (“5CSRT”)

The 5-choice serial reaction time task is a behavioral assay that is used to measure attention and impulsivity in rats. Rats are trained to respond correctly to aperture illuminations reinforced to food rewards until they reach >70% correct responses during a 30-minute baseline session. Once animals reach baseline criteria, a variable stimulus duration (SD) and variable inter-trial interval (ITI) test can be used to manipulate normal baseline performance. When the SD is decreased the number of correct responses, a measure of attention, is significantly decreased. When the ITI is increased from that used during baseline training impulsivity, as measured by premature responses, is increased. Compounds and manipulations that improve attention will improve performance and those that decrease impulsivity will decrease premature responses.

5CSRT: The test apparatus consisted of ten 25×25 cm aluminum chambers (Med Associates Inc., St. Albans, Vt.). The rear wall of each chamber was concavely curved, containing five accessible apertures, each 2.5 cm square, and 4 cm deep positioned 2 cm above floor level. Standard 3-watt LEDs located at the rear of each aperture served as stimulus lights. Opposite to the apertures was the magazine dispenser, into which food rewards were deposited. The ten chambers were individually housed within sound-attenuating cabinets and were ventilated by low-level noise fans, which also served to mask extraneous background noise. Each chamber was illuminated by a 3W house-light mounted in the center of the roof alongside a small general-purpose loud speaker. The program controlling the software was developed by Conclusive Solutions (UK).

5CSRT Training: Prior to drug treatments, rats were trained to discriminate a brief visual stimulus light presented randomly in one of the five spatial locations. At the beginning of each test session, the house light was illuminated and free delivery of a single food pellet was dispensed to the food magazine. Trial initiation was triggered when the rat opened the magazine to collect this pellet. After a fixed 5 second inter-trial interval (ITI), the light at the rear of one of the five apertures was illuminated for 500 milliseconds. A single nose-poke in this opening during the period of illumination (signal period), or during the 5 second afterwards (limited hold) was reinforced by the delivery of a food pellet and a correct response was recorded. A response in a non-illuminated opening during the signal period (incorrect response) and failures to respond within the limited hold period (missed trial) were followed by a period of darkness (time out) and no food pellet was delivered. Premature responses, defined as nose-pokes into apertures during the ITI period prior to illumination, reset the ITI. When animals had been trained to perform at a level of 75% correct on the standard procedure (500 millisecond light duration, 5 second ITI) testing began in the ITI schedule where animals were exposed to variable inter-trial interval (ITI) durations (10, 7, 5, and 4 seconds). Equal numbers of each of the 4 ITIs were randomly presented during the 100 trial session. A within subjects design was used such that all animals received all treatments in a fully counterbalanced regimen. All subjects received drug treatments and testing was twice a week giving a minimum of 2 days washout period. Interspersed with those treatment days were standard training days (500 ms stimulus/5 second ITI) reinstating baseline performance until all subjects were back to >75% performance on the percent correct responding. Using a Latin-square dosing design in which each rat receives each dose of the compound, the ITI data was collected over a 10-day testing period.

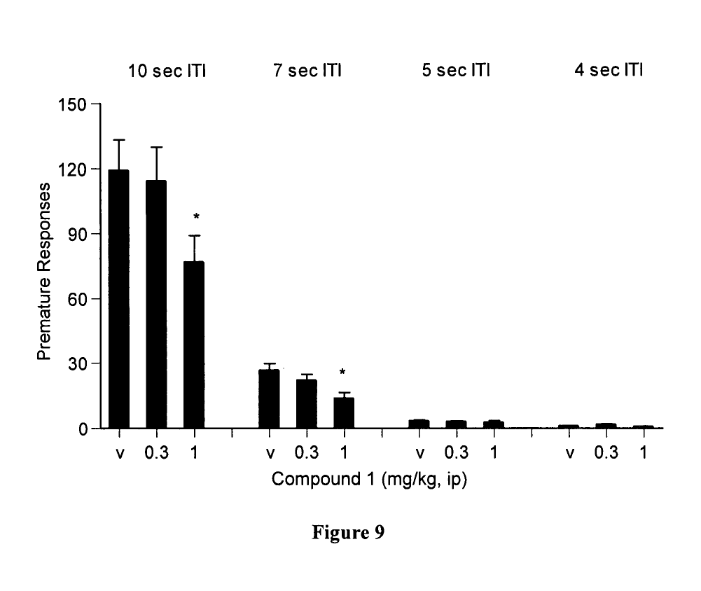

Results for Compound 1 are depicted in FIG. 9. Compound 1 produced a dose-dependent decrease in premature responding under the longer ITI conditions (10 and 7 sec) with a dose of 1 mg/kg, ip producing a statistically significant decrease in premature responding (p<0.05 vs vehicle in the 10 and 7 sec ITI conditions)

### CONCLUSION

Compound 1 decreases premature responding in the 5-choice serial reaction time task indicative of efficacy in impulsivity disorders.

We claim:

1. A method of treating a cognitive disorder in a patient, comprising administering to said patient a therapeutically effective amount of a compound of formula I: 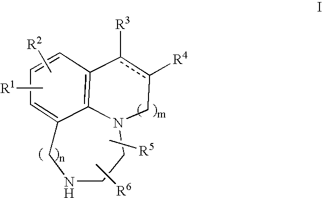
or a pharmaceutically acceptable salt thereof, wherein: [symbol]
designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R.

2. The method according to claim 1, wherein [symbol] designates a single bond.

3. The method according to claim 2, wherein:
R¹ is R, OR, halogen, cyano, or —C₁₋₃ perfluoroalkyl; and
R² is R, OR, halogen, cyano, or —C₁₋₃ perfluoroalkyl.

4. The method according to claim 3, wherein at least one of R¹ and R² is —OH.

5. The method according to claim 3, wherein R³ and R⁴ are taken together, with the carbon atoms to which they are bound, to form a saturated or unsaturated 5-8 membered ring, wherein said ring is optionally substituted with 1-3 groups independently selected from halogen, —R, or OR.

6. The method according to claim 1, wherein said compound is of formula I-a or I-b: 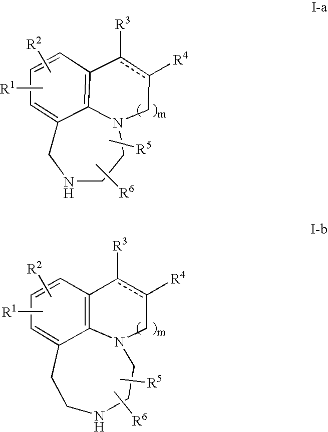
or a pharmaceutically acceptable salt thereof.

7. The method according to claim 1, wherein said compound is of formula I-c or I-d: 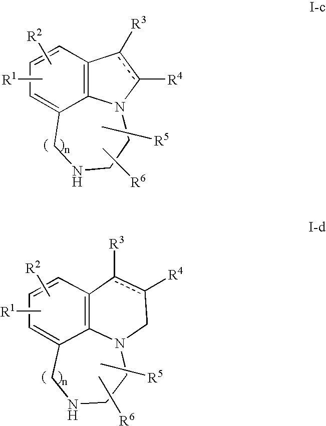
or a pharmaceutically acceptable salt thereof.

8. The method according to claim 7, wherein said compound is of formula II or III: 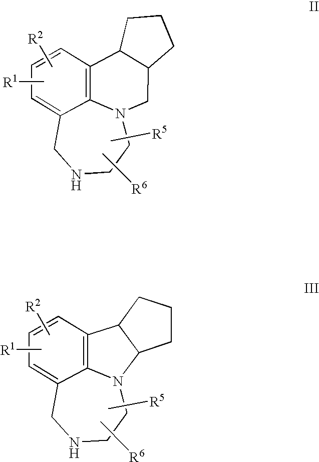
or a pharmaceutically acceptable salt thereof.

9. The method according to claim 1, wherein said compound is of formula I-e or I-f: 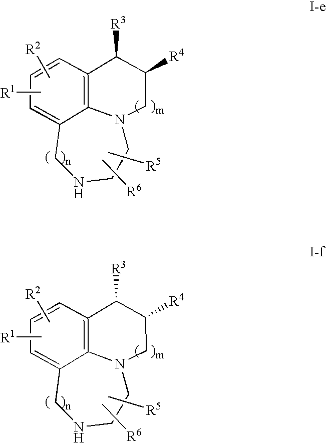
or a pharmaceutically acceptable salt thereof.

10. The method according to claim 9, wherein said compound is of formula IV or V: 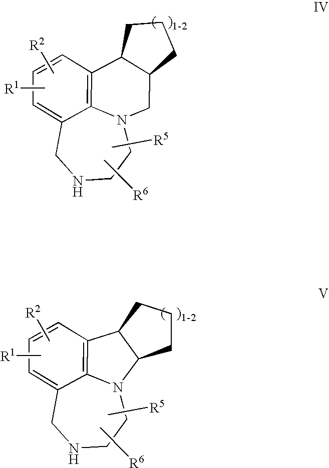
or a pharmaceutically acceptable salt thereof.

11. The method according to claim 1, wherein said compound is selected from:
2-bromo-4,5,6,7,9,9a,10,11,12,12a-decahydrocyclopenta[c][1,4]diazepino [6,7,1-ij]quinoline;
2-bromo-4,5,6,7,9,9a,10,11,12,13,14,14a-dodecahydrocyclohepta[c][1,4 ]diazepino[6,7,1-ij]quinoline;
2-chloro-4,5,6,7,9,9a,10,11,12,12a-decahydrocyclopenta[c][1,4]diazepino [6,7,1-ij]quinoline;
2-chloro-4,5,6,7,9,9a,10,11,12,13,14,14a-dodecahydrocyclohepta[c][1,4]diazepino[6,7,1-ij]quinoline;
2-phenyl-4,5,6,7,9,9a,10,11,12,12a-decahydrocyclopenta[c][1,4]diazepino [6,7,1-ij]quinoline;
2-methoxy-4,5,6,7,9,9a,10,11,12,12a-decahydrocyclopenta[c][1,4]diazepino [6,7,1-ij]quinoline;
1-fluoro-4,5,6,7,9,9a,10,11,12,12a-decahydrocyclopenta[c][1,4]diazepino [6,7,1-ij]quinoline;
1-fluoro-4,5,6,7,9,9a,10,11,12,13,14,14a-dodecahydrocyclohepta[c][1,4]diazepino[6,7,1-ij]quinoline;
1-(trifluoromethyl)-4,5,6,7,9,9a,10,11,12,12a-decahydrocyclopenta[c][1,4]diazepino[6,7,1-ij]quinoline;
1-fluoro-2-methoxy-4,5,6,7,9,9a,10,11,12,12a-decahydrocyclopenta[c][1,4]diazepino [6,7,1-ij]quinoline;
1-fluoro-2-methoxy-4,5,6,7,9,9a,10,11,12,13,14,14a-dodecahydrocyclo-hepta[c][1,4]diazepino[6,7,1-ij]quinoline;
4,5,6,7,9,9a,10,11,12,12a-decahydrocyclopenta[c][1,4]diazepino[6,7,1-ij]quinoline;
4,5,6,7,9,9a,10,11,12,13,14,14a-dodecahydrocyclohepta[c][1,4]diazepino [6,7,1-ij]quinoline;
(−)-4,5,6,7,9,9a10,11,12,12a-decahydrocyclopenta[c][1,4]diazepino[6,7,1-ij]quinoline;
(9aR, 14aS)-4,5,6,7,9,9a,10,11,12,13,14,14a-dodecahydrocyclohepta[c][1,4]diazepino[6,7,1-ij]quinoline; or
(9aS, 14aR)-4,5,6,7,9,9a,10,11,12,13,14,14a-dodecahydrocyclohepta[c][1,4]diazepino[6,7,1-ij]quinoline;
4,5,6,7,9a,10,11,12,13,13a-decahydro-9H-[1,4]diazepino[6,7,1-de]phenanthridine;
1,2,3,4,9,10-hexahydro-8H-cyclopenta[b][1,4]diazepino[6,7,-hi]indole;
1,2,3,4,8,9,10,10a-octahydro-7bH-cyclopenta[b][1,4]diazepino[6,7, 1-hi]indole;
(7bS,10aS)-1,2,3,4,8,9,10,10a-octahydro-7bH-cyclopenta[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
6-methyl-1,2,3,4,9,10-hexahydro-8H-cyclopenta[b][1,4]diazepino[6,7,1-hi]indole;
(2S)-(rel-7bR,10aR)-2-methyl-1,2,3,4,8,9,10,10a-octahydro-7bH-cyclopenta[b][1,4]diazepino[6,7,1-hi]indole;
(2S)-(rel-7bR,10aR)-2-methyl-1,2,3,4,8,9,10,10a-octahydro-7bH-cyclopenta[b][1,4]diazepino[6,7,1-hi]indole;
(2S)-(rel-7bS,10aS)-2-methyl-1,2,3,4,8,9,10,10a-octahydro-7bH-cyclopenta[b][1,4]diazepino[6,7, 1-hi]indole;
(2R)-(rel-7bR,10aR)-2-methyl-1,2,3,4,8,9,10,10a-octahydro-7bH-cyclopenta[b][1,4]diazepino[6,7,1-hi]indole;
(2R)-(rel-7bR,10aR)-2-methyl-1,2,3,4,8,9,10,10a-octahydro-7bH-cyclopenta[b ][1 ,4]diazepino[6,7, 1-hi]indole;
(2R)-(rel-7bS,10aS)-2-methyl-1,2,3,4,8,9,10,10a-octahydro-7bH-cyclopenta[b][1,4]diazepino[6,7,1-hi]indole;
rel-(4S,7bS,10aS)-4-methyl-1,2,3,4,8,9,10,10a-octahydro-7bH-cyclopenta[b][1,4]diazepino[6,7,1-hi]indole;
rel-(4S,7bS,10aS)-4-methyl-1,2,3,4,8,9,10,10a-octahydro-7bH-cyclopenta[b]-[1,4]diazepino[6,7,1-hi]indole;
rel-(4R,7bS, 10aS)-4-methyl-1,2,3,4,8,9,10,10a-octahydro-7bH-cyclopenta[b][1,4]diazepino [6,7,1-hi]indole;
9-methyl-1,2,3,4,8,9,10,10a-octahydro-7bH-cyclopenta[b][1,4]diazepino[6,7,1-hi]indole;
(7bR,9R,10aR)-9-methyl-1,2,3,4,8,9,10,10a-octahydro-7bH-cyclopenta[b][1,4]diazepino[6,7,1-hi]indole;
9,9-dimethyl-1,2,3,4,8,9,10,10a-octahydro-7bH-cyclopenta[1,4]diazepino[6,7,1-hi]indole;
(7bR,10aR)-9,9-dimethyl-1,2,3,4,8,9,10,10a-octahydro-7bH-cyclopenta[b][1,4]diazepino[6,7,1-hi]indole; and
(7bS,10aS)-9,9-dimethyl-1,2,3,4,8,9,10,10a-octahydro-7bH-cyclopenta[b][1,4]diazepino[6,7,1-hi]indole;
or a pharmaceutically acceptable salt thereof.

12. The method of claim 11, wherein said compound is the hydrochloride salt.

13. The method of claim 1, wherein the cognitive disorder is ADD or ADHD.

14. The method of claim 13, wherein the mammal is a pediatric patient.

15. The method of claim 1, wherein the cognitive disorder is a learning disorder.

16. The method of claim 15, wherein the learning disorder is autism, dyslexia, Asperger's Syndrome, a specific learning disability, dysgraphia, dyscalculia, dyspraxia, visual perceptual deficit, or auditory perceptual deficit.

17. The method of claim 1, wherein the cognitive disorder is an impulsivity disorder.

18. The method of claim 17, wherein the impulsivity disorder is borderline personality disorder, a disruptive behavior disorder, an impulse control disorders, or Tourette's Syndrome.

19. The method of claim 1, wherein the cognitive disorder is a behavioral addiction or addictive disorder.

20. The method of claim 19, wherein the behavioral addiction or addictive disorder is gambling, sex addiction, eating disorders, spending addiction, rage/anger, workaholism, exercise addiction, a risk taking addiction, or perfectionism.

21. The method of claim 1, further comprising administering an additional therapeutic agent selected from an acetylcholinesterase inhibitor, galantamine, a neuroprotective agent, a therapeutic agent for treating ADD/ADHD.

22. The method of claim 21, wherein the additional agent is selected from donepezil hydrochloride, galantamine, memantine, methylphenidate, atomoxetine, or amphetamine/dextroamphetamine.

23. A method for treating PMS or PMDD, or one or more symptoms associated with PMS or PMDD, in a patient, comprising administering to said patient a therapeutically effective amount of a compound of formula I: 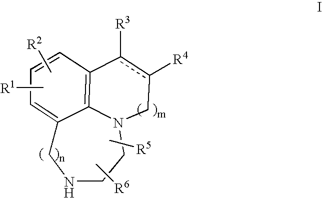
or a pharmaceutically acceptable salt thereof, wherein: [symbol]
designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R.

24. The method of claim 23, wherein said symptom is one or more of irritability, depressed mood, anxiety, sleep disturbance, difficulty concentrating, angry outbursts, breast tenderness and bloating.

24. The method of claim 23, further comprising administering to said patient a selective serotonin reuptake inhibitor.

25. The method of claim 24, wherein said selective serotonin reuptake inhibitor is fluoxetine, venlafaxine, paroxetine, duloxetine, or sertraline.
